(19) United States
(12) Patent Application Publication
(10) Pub. No.: US 2024/0173529 A1
(43) Pub. Date: May 30, 2024

(54) CONNECTOR

(71) Applicant: TERUMO MEDICAL PRODUCTS (HANGZHOU) CO., LTD., Hangzhou, Zhejiang (CN)

(72) Inventors: Hongyuan HU, Hangzhou (CN); Binhua YAN, Hangzhou (CN); Juan ZHANG, Hangzhou (CN); Dianhong CHEN, Hangzhou (CN)

(73) Assignee: TERUMO CORPORATION, Tokyo (JP)

(21) Appl. No.: 18/551,805

(22) PCT Filed: Apr. 25, 2022
(86) PCT No.: PCT/CN2022/088878

§ 371 Date: Sep. 21, 2023

(30) Foreign Application Priority Data
Apr. 25, 2021 (CN) 202110451189.6

(51) Int. Cl. A61M 39/10; A61M 39/00

(52) U.S. Cl. CPC A61M 39/105; A61M 2039/0018

(57) ABSTRACT

A connector, comprising: a housing which has a first port, a second port, and a first chamber in communication with the first port and the second port; a cover, which is connected to the housing and which has a second chamber in communication with the first chamber; and a bulging portion, which is disposed in the first chamber and protrudingly extends toward the second chamber. A side groove which obliquely curves and extends towards the second chamber is provided in the first chamber. The groove structure of the connector can guide a portion of fluid which is flowing into the first chamber from the first port towards the second chamber, so as to form a spiral upward vortex, thereby enlarging a flushing region, increasing the flushing efficiency, and reducing the risk of pollution.

This application claims the benefit of priority to Chinese patent application No. 202110451189.6, filed on Apr. 25, 2021, entitled “CONNECTOR”, the entire disclosures of which are incorporated herein by reference.

### TECHNICAL FIELD

The present disclosure relates to a connector, and more particularly to a connector suitable for communicating a plurality of catheters with each other in a liquid conveying pipeline.

### BACKGROUND

When performing infusion, blood transfusion, or blood collection operations on patients, in order to achieve therapeutic effects, it is necessary to connect a plurality of catheters to form an infusion pipeline from a drug solution (or blood) to a patient, and a plurality of infusion pipelines can be connected with each other through connectors.

For example, in order to collect blood samples from patients during blood pressure monitoring, a connector is usually added to a monitoring pipeline, and the blood collection operation is carried out after connecting the connector with a syringe. After the blood collection is completed, in order to continue blood pressure monitoring and prevent the blood from clotting in the pipeline, it is necessary to flush the pipeline (including the connector) to remove the blood. A typical method is to flush a downstream pipeline with a certain pressure and amount of flushing fluid flowing from an upstream flushing bag into a human body. However, due to a small amount of the flushing fluid during flushing, an existing connector structure allows most of the flushing fluid to rush towards a top located diagonally in a connector cavity after entering the connector cavity. However, the top in the connector cavity on a side where the flushing fluid enters is difficult to flush by the flushing fluid, forming a dead corner for the blood or drug solution residue, which leads to safety hazards.

### SUMMARY

Embodiments of the present disclosure provide a connector, which can effectively guide a flushing fluid into the connector, increase a fluid velocity entering the connector, and improve a flushing effect of the flushing fluid.

According to an aspect of the present disclosure, a connector is provided. The connector includes: a housing having a first port for a fluid to flow into the housing, a second port for the fluid to flow out of the housing and a first chamber communicating the first port with the second port; a cover connected with the housing, wherein the cover has a third port for the fluid to flow into the housing and a second chamber communicated with the first chamber; and a bulging portion arranged in the first chamber and protrudes towards the second chamber, wherein the bulging portion has a first side close to the first port and a second side close to the second port, and the first side is configured to guide a portion of the fluid flowing into the first chamber from the first port to flow towards the second chamber; wherein an inner wall of the first chamber is provided with a side groove extending obliquely and curvedly towards the second chamber, and the side groove is arranged between the first port and the first side of the bulging portion and configured to guide the portion of the fluid flowing into the first chamber from the first port to flow towards the second chamber.

According to some embodiments, the first side of the bulging portion is provided with an intermediate groove extending towards an axial central portion of the first chamber and having a gradually reduced depth.

According to some embodiments, the inner wall of the first chamber includes a bottom wall and a circumferential side wall, and the side groove extends obliquely and curvedly along the circumferential side wall from the bottom wall of the first chamber to an upper edge of the circumferential side wall.

According to some embodiments, the first chamber is provided with an inflow space communicating the first port with the intermediate groove and communicating the first port with the side groove, and a flow path cross-sectional area of the inflow space is smaller than a flow path cross-sectional area of the first port.

According to some embodiments, a top of the bulging portion is higher than an apex of the first port.

According to some embodiments, the side groove accounts for 15% to 35% of a circumference of the circumferential side wall along a circumferential direction of the circumferential side wall.

According to some embodiments, a ratio between a width of the side groove and a radius of the first port ranges from 2:5 to 3:5.

According to some embodiments, a depth of the side groove is gradually reduced with the side groove extending obliquely and curvedly towards the second chamber, and the fluid flowing out of the side groove forms an upward spiral vortex to force the fluid flowing out of the intermediate groove to spiral upward.

According to some embodiments, a ratio between a maximum depth of the side groove and a radius of the first port ranges from 3:1 to 4:1.

According to some embodiments, the intermediate groove has an inlet adjacent to the first port and an outlet adjacent to a top of the bulging portion, and a distance between the inlet and a lowest point of the first port is less than or equal to a radius of the first port.

According to some embodiments, a projection of a center line of the intermediate groove in a horizontal plane where a center line of the first port is located is offset relative to the center line of the first port towards the side groove.

According to some embodiments, an offset distance between the projection of the center line of the intermediate groove in the horizontal plane where the center line of the first port is located and the center line of the first port is less than or equal to twice a width of the intermediate groove.

According to some embodiments, a ratio between a width of the intermediate groove and the radius of the first port ranges from 1:5 to 1:1.

According to some embodiments, a ratio between a horizontal distance from the outlet of the intermediate groove to the first port and the radius of the first port ranges from 2:1 to 4:1.

The technical solution of the embodiments of the present disclosure has following beneficial effects:

According to some embodiments, an inner wall of the first chamber is provided with a side groove extending obliquely and curvedly towards the second chamber, and the side groove is arranged between the first port and the first side of the bulging portion and configured to guide a portion of the fluid flowing into the first chamber from the first port towards the second chamber. When using a fluid such as a flushing fluid to flush the connector, after the flushing fluid enters the first chamber through the first port, some of the flushing fluid flows obliquely upward through the first side of the bulging portion, and some of the flushing fluid flows obliquely upward through the side groove. The side groove can guide the flushing fluid to rotate upward to form a rotating vortex in the first chamber, which can increase a flowing rate of the flushing fluid entering the first chamber, thereby improving a flushing effect of the flushing fluid, and preventing bacterial infections caused by residual drug solution and blood.

According to some embodiments, the first side of the bulging portion is provided with an intermediate groove extending towards an axial central portion of the first chamber and having a gradually reduced depth. The inner wall of the first chamber includes a bottom wall and a circumferential side wall, and the side groove extends obliquely and curvedly along the circumferential side wall from the bottom wall of the first chamber to an upper edge of the circumferential side wall, which can improve the flowing rate of the flushing fluid and further improve the flushing effect of the flushing fluid.

According to some embodiments, the first chamber is provided with an inflow space communicating the first port with the intermediate groove and communicating the first port with the side groove, and a flow path cross-sectional area of the inflow space is smaller than a flow path cross-sectional area of the first port. The inflow space can gather the flushing fluid, improve the flowing rate of the flushing fluid, and further improve the flushing effect of the flushing fluid.

According to some embodiments, the connector has a specific groove structure, where the side groove and the intermediate groove can increase the flowing rate of the flushing fluid, expand the flushing area, and especially improve the flushing effect at the corners.

### BRIEF DESCRIPTION OF THE DRAWINGS

Other features and advantages of the present disclosure will be better understood from the following preferred embodiments described in detail with reference to the accompany drawings, where the same signs represent the same or similar components, in which:

FIG. 1 shows a perspective view of a connector according to an embodiment of the present disclosure;

FIG. 2 shows a cross-sectional view of a connector according to an embodiment of the present disclosure;

FIG. 3 shows a partial perspective view of a housing of a connector according to an embodiment of the present disclosure;

FIG. 4 shows a partial cross-sectional view of a housing of a connector according to an embodiment of the present disclosure;

FIG. 5 shows a partial perspective view of a housing of a connector according to still another embodiment of the present disclosure;

FIG. 6 shows a partial cross-sectional view of a housing of a connector according to still another embodiment of the present disclosure;

FIG. 7 shows a partial perspective view of a housing of a connector according to yet another embodiment of the present disclosure; and

FIG. 8 shows a partial cross-sectional view of a housing of a connector according to yet another embodiment of the present disclosure.

### DETAILED DESCRIPTION

The implementation and application of specific embodiments will be discussed in detail below. However, it should be understood that the specific embodiments discussed only exemplarily illustrate specific ways of implementing and applying the present disclosure, and are not limiting the scope of the present disclosure. The description of structural positions of each component, such as up, down, top, bottom, and other directions, is not absolute, but relative. When the components are arranged as shown in the figures, these directional representations are appropriate, but when the positions of the components in the figures change, these directional representations also change accordingly.

The volume of a cavity in the existing connectors is larger than that of a previous pipeline, resulting in a rapid decrease in the flowing rate of the flushing liquid after entering the cavity of the connectors, thus it is difficult to flush residual blood in certain corners. Therefore, an embodiment of the present disclosure provides a connector, which can effectively guide the flushing liquid into the connector, increase the flowing rate of the flushing liquid entering the connector, and improve the flushing effect of the flushing liquid. According to the present disclosure, the flushing liquid may include but is not limited to reagents, flushing reagents, and water.

According to some embodiments of the present disclosure, the connector includes: a housing having a first port for a fluid to flow into the housing, a second port for the fluid to flow out of the housing, and a first chamber communicating the first port with the second port; a cover connected with the housing, wherein the cover has a third port for the fluid to flow into the housing and a second chamber communicated with the first chamber; and a bulging portion arranged in the first chamber and protrudes towards the second chamber, wherein the bulging portion has a first side close to the first port and a second side close to the second port, and the first side is configured to guide a portion of the fluid flowing into the first chamber from the first port to flow towards the second chamber; wherein an inner wall of the first chamber is provided with a side groove extending obliquely and curvedly towards the second chamber, and the side groove is arranged between the first port and the first side of the bulging portion and configured to guide the portion of the fluid flowing into the first chamber from the first port to flow towards the second chamber.

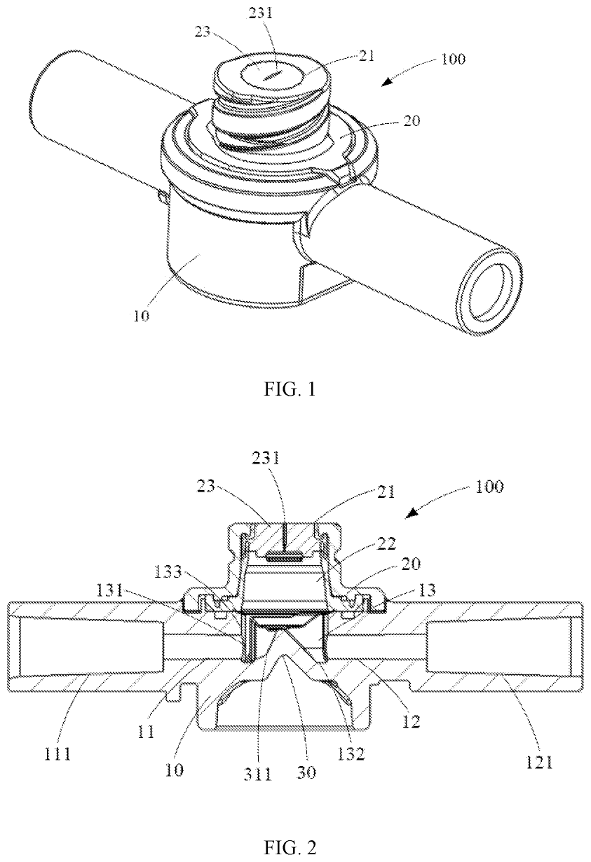

FIGS. 1 and 2 illustrate a schematic view of a connector 100 according to an embodiment of the present disclosure. The connector 100 includes a housing 10 and a cover 20. The housing 10 and the cover 20 may be connected with each other through methods known in the art. The housing 10 and the cover 20 may be formed using resin materials, including but not limited to polyethylene, polypropylene, polyolefin such as ethylene vinyl-acetate copolymer, polyurethane, polyamide, polyester, polycarbonate, polybutadiene and polyvinyl chloride.

The housing 10 has an opening or a first port 11 for a fluid to flow into the housing, an opening or a second port 12 for the fluid to flow out of the housing, and a first chamber 13 communicating the first port 11 with the second port 12. The first port 11 and the second port 12 may have the same shape and size. The cover 20 has an opening or a third port 21 for the fluid to flow into the housing and a second chamber 22 in the third port 21. The second chamber 22 is communicated with the first chamber 13. The fluid flowing through the first port 11 can be the same as or different from the fluid flowing through the third port 21.

In a specific application, the connector 100 is used in an infusion pipeline for infusion of patients to connect multiple catheters with each other. The first port 11 is connected with a first catheter that forms a main pipeline, the second port 12 is connected with a second catheter that forms the same main pipeline, and the third port 21 is connected with a third catheter that forms another pipeline. For example, the first port 11 and the second port 12 extend in opposite directions from two sides of the housing 10, and the axes of the first port 11 and the second port 12 are aligned with each other and arranged in a straight line.

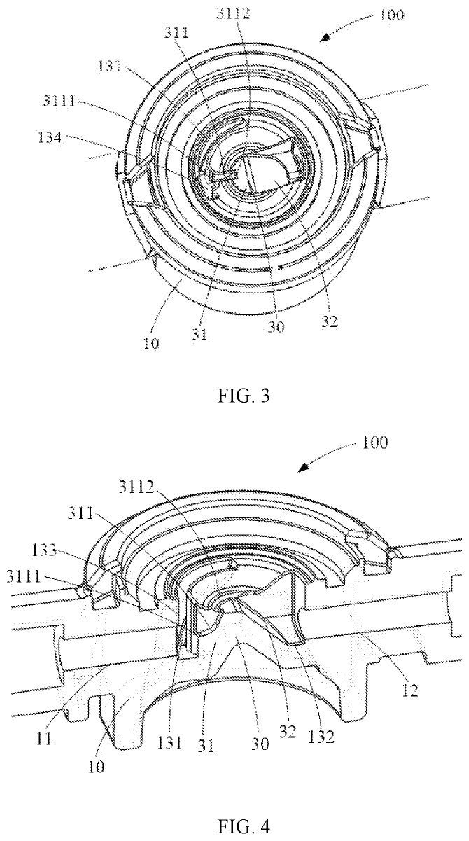

With reference to FIGS. 3 and 4, the connector further includes a bulging portion 30. The bulging portion 30 is arranged on an axial central portion in the first chamber 13 and protrudes towards the second chamber 22. The bulging portion 30 has a first side 31 close to the first port 11 and a second side 32 close to the second port 12. The first side 31 is configured to guide a portion of the fluid flowing into the first chamber 13 from the first port 11 towards the second chamber 22.

The first side 31 of the bulging portion 30 has an intermediate groove 311 extending towards the axial central portion of the first chamber 13 with a gradually decreasing depth. An inner wall of the first chamber 13 is provided with a side groove 131 that extends obliquely and curvedly towards the second chamber 22. The side groove 131 is arranged between the first port 11 and the first side 31 of the bulging portion 30, and is communicated with the first port 11 and the second chamber 22. The side groove 131 can guide a portion of the fluid flowing from the first port 11 into the first chamber 13 towards the second chamber 22.

The blood or drug solution is prone to remain in a top of the second chamber, but the structure of the existing connector allows most of the flushing liquid to rush towards the top located diagonally in the second chamber after entering the first chamber. However, the top of the side where the flushing liquid enters the second chamber is difficult to be flushed by the flushing liquid, forming a dead corner for the blood or drug solution residue. According to the embodiment of the present disclosure, the connector is provided with a groove structure, in particular, the side groove extends from a bottom of the first chamber obliquely and curvedly upward to guide the flushing liquid to spiral upward, which can successfully form a rotating eddy current in the chamber of the connector to flush the entire top of the second chamber, and prevent residual blood or drug solution from remaining in the second chamber and causing bacterial infections.

The inner wall of the first chamber 13 has a bottom wall 132 and a circumferential side wall 133, and the side groove 131 extends obliquely and curvedly upward along the circumferential side wall 133 from the bottom wall 132 of the first chamber 13 to an upper edge of the circumferential side wall 133. The first port 11 and the second port 12 are formed on the circumferential side wall 133. In the embodiment shown in FIG. 2, an end of the first port 11 is provided with a first conical opening 111, and an end of the second port 12 is provided with a second conical opening 121. A radius of the first conical opening 111 gradually increases outwards in a direction away from the first port 11, and a radius of the second conical opening 121 gradually increases outwards in a direction away from the second port 12, thereby facilitating the connection between the first port 11, the second port 12, and other catheters.

In some embodiments, the first chamber 13 is provided with an inflow space 134. The inflow space 134 communicates the first port 11 with the intermediate groove 31 and communicates the first port 11 with the side groove 311, and a flow path cross-sectional area of the inflow space 134 is smaller than a flow path cross-sectional area of the first port 11. The inflow space 134 can gather the flushing fluid, improve the flowing rate of the flushing fluid, and further improve the flushing effect of the flushing fluid.

In some embodiments, the top of the bulging portion 30 is higher than an apex of the first port 11, that is, the bulging portion 30 protrudes upward from the bottom wall 132 over a penetration position of the flow path of the first port 11 and the second port 12, allowing the flushing fluid entering the first chamber 13 of the connector 100 through the first port 11 to fully impact the bulging portion 30, thereby increasing the flowing rate of the flushing fluid and improving the flushing effect.

In some embodiments, the side groove 131 accounts for 15% to 35% of a circumference of the circumferential side wall 133 along the circumferential direction of the circumferential side wall 133. In other words, when viewed above the connector 100, the side groove 131 extends 0.15 to 0.35 circles of the circumference of the circumferential side wall 133 along the circumferential side wall 133. In some embodiments, a ratio between a width of the side groove 131 and a radius of the first port 11 ranges from 2:5 to 3:5. In some embodiments, a depth of the side groove 131 is gradually reduced with the side groove 131 extending obliquely and curvedly upward from the bottom of the first chamber 13. In some embodiments, the ratio between a maximum depth of the side groove 131 and the radius of the first port 11 ranges from 3:1 to 4:1.

In some embodiments, the intermediate groove 311 has an inlet 3111 adjacent to the first port 11 and an outlet 3112 adjacent to the top of the bulging portion 30. A distance between the inlet 3111 and a lowest point of the first port 11 is less than or equal to the radius R of the first port. In some embodiments, a position of the inlet 3111 of the intermediate groove 311 is flush with the lowest point of the first port 11. In other embodiments, the position of the inlet 3111 of the intermediate groove 311 is flush with a center position of the first port 11.

In other embodiments, the distance between the inlet 3111 of the first port 11 and the lowest point of the first port 11 ranges from 0 to 1.8 R.

In a horizontal plane where a center line of the first port 11 is located, a projection of a center line of the intermediate groove 311 is offset relative to the center line of the first port 11. Specifically, the center line of the intermediate groove 311 is offset relative to the center line of the first port 11 towards the side slot 113, and an offset distance is less than or equal to twice the width of the intermediate groove 311, or may be equal to zero, that is, the projection of the center line of the intermediate groove 311 is aligned with the center line of the first port 11.

In some embodiments, the offset distance of the intermediate groove 311 relative to the center line of the first port 11 towards the side groove 113 is equal to half of the width of the intermediate groove 311. By setting the intermediate groove 311 to offset towards the side groove 113, the intermediate groove 311 and the side groove 113 jointly act on the fluid, causing the fluid passing through the side groove 113 to force the fluid in the intermediate groove 311 to forming a positive vortex together. All the fluid enters the top of the second chamber to flush the entire top of the second chamber, including the corners, to prevent residual drug solution and blood from being trapped in the second chamber and causing bacterial infections.

In some embodiments, the ratio between the width of the intermediate groove 311 and the radius of the first port 11 ranges from 1:5 to 1:1. In some embodiments, a horizontal distance between the outlet 3112 of the intermediate groove 311 and the first port 11 is 2 to 4 times the radius of the first port 11.

In some embodiments, the third port 21 of the cover 20 is provided with an elastic valve core 23. The elastic valve core 23 has a slit 231, and the third port 21 can be connected or disconnected by opening or closing the slit 231. For example, when the catheter is inserted through the third port 21, the elastic valve core 23 can be deformed elastically to open the slit 231 so as to allow the catheter to be connected to the connector 100. The cover 20 can also include other components, and the structure of the cover 20 is known to those skilled in the art, which will not be repeated here.

The elastic valve core 23 can be formed from elastic materials, including but not limited to synthetic rubber such as polybutadiene, nitrile, chloroprene, natural rubber such as polyisoprene, or thermosetting elastomers such as polyurethane rubber, silicone rubber, fluorine rubber, thermoplastic elastomers, or other elastomers.

In the embodiment shown in FIGS. 3 and 4, the radii of the first port 11 and the second port 12 are R, the position of the inlet 3111 of the intermediate groove 311 is flush with the center position of the first port 11, the width W of the intermediate groove 311 is 0.42 R, the horizontal distance between the outlet 3112 of the intermediate groove 311 and the first port 11 is 2.6 R, and the projection of the center line of the intermediate groove 311 in the horizontal plane where the center line of the first port 11 is located is offset relative to the center line of the first port 11 towards the side groove 131, and the offset distance is 0.5 W. The side groove 131 accounts for 22% of the circumference of the circumferential side wall 133 along the circumferential direction of the circumferential side wall 133. The width of the side groove 131 is 0.52 R, and the maximum depth of the side groove 131 is 4 R.

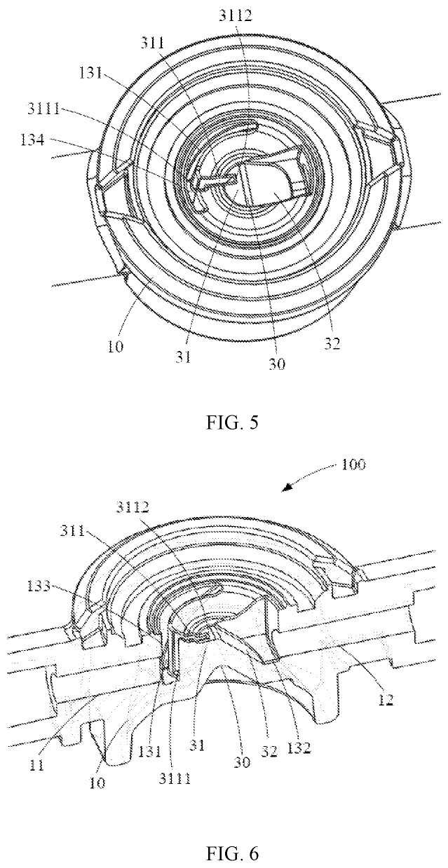

In the embodiment shown in FIGS. 5 and 6, the radii of the first port 11 and the second port 12 are R, the position of the inlet 3111 of the intermediate groove 311 is flush with the center position of the first port 11, the width W of the intermediate groove 311 is 0.36 R, the horizontal distance between the outlet 3112 of the intermediate groove 311 and the first port 11 is 2.8 R, and the projection of the center line of the intermediate groove 311 in the horizontal plane where the center line of the first port 11 is located is offset relative to the center line of the first port 11 towards the side groove 131, and the offset distance is 1.3 W. The side groove 131 accounts for 24% of the circumference of the circumferential side wall 133 along the circumferential direction of the circumferential side wall 133. The width of the side groove 131 is 0.52 R, and the maximum depth of the side groove 131 is 4 R.

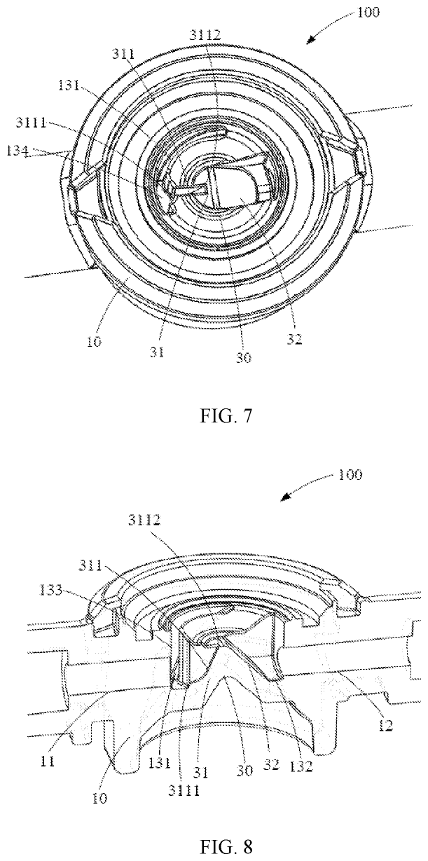

In the embodiment shown in FIGS. 7 and 8, the radii of the first port 11 and the second port 12 are R, the position of the inlet 3111 of the intermediate groove 311 is flush with the center position of the first port 11, the width W of the intermediate groove 311 is 0.42 R, the horizontal distance between the outlet 3112 of the intermediate groove 311 and the first port 11 is 3 R, and the projection of the center line of the intermediate groove 311 in the horizontal plane where the center line of the first port 11 is located is aligned with the center line of the first port 11. The side groove 131 accounts for 24% of the circumference of the circumferential side wall 133 along the circumferential direction of the circumferential side wall 133. The width of the side groove 131 is 0.52 R, and the maximum depth of the side groove 131 is 4 R.

In the embodiments shown in FIGS. 3 and 4, FIGS. 5 and 6, and FIGS. 7 and 8, the fluid, such as the flushing fluid, flows into the first chamber through the first port, a portion of the flushing fluid flows out through the intermediate groove and a portion of the flushing fluid flows out through the side groove. Due to a slope design of both the intermediate groove and the side groove from deep to shallow, the flowing rate and direction are changed. After flowing out through the side groove, the fluid forms an upward spiral vortex. Due to the viscosity of the fluid, the fluid forces the fluid flowing out of the intermediate groove to spiral upward together. The combined action of the two fluids achieves maximum flush of the interior of the connector, especially the entire top of the second chamber, thus achieving the best flushing effect and preventing residual drug solution and blood from being trapped inside and causing bacterial infections.

Although the present disclosure has been disclosed above, the present disclosure is not limited thereto. Any changes and modifications may be made by those skilled in the art without departing from the spirit and scope of the present disclosure, and the scope of the present disclosure should be determined by the appended claims.

1. A connector, comprising:
a housing having a first port for a fluid to flow into the housing, a second port for the fluid to flow out of the housing and a first chamber communicating the first port with the second port;
a cover connected with the housing, wherein the cover has a third port for the fluid to flow into the housing and a second chamber communicated with the first chamber; and
a bulging portion arranged in the first chamber and protrudes towards the second chamber, wherein the bulging portion has a first side close to the first port and a second side close to the second port, and the first side is configured to guide a portion of the fluid flowing into the first chamber from the first port to flow towards the second chamber;
wherein an inner wall of the first chamber is provided with a side groove extending obliquely and curvedly towards the second chamber, and the side groove is arranged between the first port and the first side of the bulging portion and configured to guide the portion of the fluid flowing into the first chamber from the first port to flow towards the second chamber.

2. The connector according to claim 1, wherein the first side of the bulging portion is provided with an intermediate groove extending towards an axial central portion of the first chamber and having a gradually reduced depth.

3. The connector according to claim 2, wherein the inner wall of the first chamber comprises a bottom wall and a circumferential side wall, and the side groove extends obliquely and curvedly along the circumferential side wall from the bottom wall of the first chamber to an upper edge of the circumferential side wall.

4. The connector according to claim 3, wherein the first chamber is provided with an inflow space communicating the first port with the intermediate groove and communicating the first port with the side groove, and a flow path cross-sectional area of the inflow space is smaller than a flow path cross-sectional area of the first port.

5. The connector according to claim 3, wherein a top of the bulging portion is higher than an apex of the first port.

6. The connector according to claim 3, wherein the side groove accounts for 15% to 35% of a circumference of the circumferential side wall along a circumferential direction of the circumferential side wall.

7. The connector according to claim 1, wherein a ratio between a width of the side groove and a radius of the first port ranges from 2:5 to 3:5.

8. The connector according to claim 2, wherein a depth of the side groove is gradually reduced with the side groove extending obliquely and curvedly towards the second chamber, and the fluid flowing out of the side groove forms an upward spiral vortex to force the fluid flowing out of the intermediate groove to spiral upward.

9. The connector according to claim 8, wherein a ratio between a maximum depth of the side groove and a radius of the first port ranges from 3:1 to 4:1.

10. The connector according to claim 2, wherein the intermediate groove has an inlet adjacent to the first port and an outlet adjacent to a top of the bulging portion, and a distance between the inlet and a lowest point of the first port is less than or equal to a radius of the first port.

11. The connector according to claim 10, wherein a projection of a center line of the intermediate groove in a horizontal plane where a center line of the first port is located is offset relative to the center line of the first port towards the side groove.

12. The connector according to claim 11, wherein an offset distance between the projection of the center line of the intermediate groove in the horizontal plane where the center line of the first port is located and the center line of the first port is less than or equal to twice a width of the intermediate groove.

13. The connector according to claim 10, wherein a ratio between a width of the intermediate groove and the radius of the first port ranges from 1:5 to 1:1.

14. The connector according to claim 10, wherein a ratio between a horizontal distance from the outlet of the intermediate groove to the first port and the radius of the first port ranges from 2:1 to 4:1.
